(19) United States
(12) Patent Application Publication
(10) Pub. No.: US 2017/0014422 A1
(43) Pub. Date: Jan. 19, 2017

(54) PHARMACEUTICAL COMPOSITION COMPRISING IVABRADINE HYDROCHLORIDE POLYMORPH IV

(71) Applicant: SYNTHON B.V., Nijmegen (NL)

(72) Inventors: LISA BAKKER-HOLMDAHL, NIJMEGEN (NL); DEEPAK MURPANI, NIJMEGEN (NL)

(73) Assignee: SYNTHON B.V., NIJMEGEN (NL)

(21) Appl. No.: 15/116,467

(22) PCT Filed: Jul. 22, 2014
(86) PCT No.: PCT/EP2014/065724

§ 371 Date: Aug. 3, 2016

(30) Foreign Application Priority Data
Feb. 14, 2014 (EP) 14155218.2

(51) Int. Cl. A61K 31/55; A61K 9/20; A61J 1/03; A61K 9/28

(52) U.S. Cl. CPC A61K 31/55; A61K 9/284; A61K 9/2018; A61K 9/2009; A61K 9/2059; A61K 9/2095; A61J 1/035

(57) ABSTRACT

The present invention relates to stable pharmaceutical compositions comprising ivabradine hydrochloride polymorph IV, characterized in that the composition is stabilized by a moisture barrier with a WVTR of less than 0.35 g/m²/day at 38° C./90% RH. The invention further relates to the use of said composition as a medicament, particularly in the treatment of stable angina pectoris and chronic heart failure.

### BACKGROUND OF THE PRESENT INVENTION

Ivabradine, chemically 3-(3-{[((7S)-3,4-dimethoxybicyclo[4.2.0]octa-1,3,5-trien-7-yl)methyl]methylamino}propyl)-7,8-dimethoxy-1,3,4,5-tetrahydro-2H-3-benzazepin-2-one of formula (I),

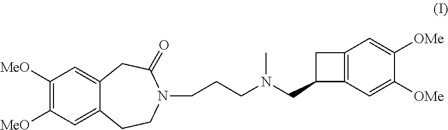 [SMILES: COc1cc2c(cc1OC)CC(=O)N(CCCN(C)C[C@H]1Cc3cc(OC)c(OC)cc31)CC2]

is a pharmaceutically active compound used for the treatment of stable angina pectoris and chronic heart failure. The compound was discovered by Adir and is disclosed in EP534859. The compound may form acid addition salts, for instance Ivabradine hydrochloride, which is the active ingredient in the medicinal product sold under the brand name Corlentor® and Procoralan® by Laboratoires Servier.

Ivabradine hydrochloride exhibits polymorphism. WO2006092493 discloses polymorph β of ivabradine hydrochloride, its process of preparation and compositions comprising this polymorph. Polymorph β, which is a tetrahydrate, is the most stable form and is present in the marketed Corlentor® and Procoralan® tablets. Other polymorphic forms of ivabradine hydrochloride are disclosed in WO2005110993, WO2006092491, WO2006092492, WO2006092494, WO2007042656, WO2007042657 and WO2013064307. The prior art thus teaches that ivabradine hydrochloride crystallizes very easily in various forms.

WO2013064307 discloses ivabradine hydrochloride polymorph IV, which is a hemihydrate. It was experienced in our laboratory that polymorph IV, especially in pharmaceutical compositions, transforms over time into the more stable polymorph β. This transformation is even more pronounced in humid environments.

Thus in view of the above, there is a need for pharmaceutical compositions comprising ivabradine hydrochloride polymorph IV, which are stable and suitable for use on a commercial scale.

### BRIEF DESCRIPTION OF THE PRESENT INVENTION

The present invention relates to a pharmaceutical composition comprising ivabradine hydrochloride polymorph IV, wherein the X-ray powder diffraction pattern of polymorph IV comprises characteristic peaks at the following 2 theta (±0.2) angles: 8.8°, 15.6°, 17.1°, 19.9°, 24.2° and 24.5°, measured using a Cu Kα radiation, characterized in that the composition is stabilized by a moisture barrier with a WVTR of less than 0.35 g/m²/day at 38° C./90% RH.

Additionally, the invention provides a process for preparing said pharmaceutical composition comprising blending ivabradine hydrochloride polymorph IV with one or more pharmaceutically acceptable excipients, followed by compressing the blend into tablets.

Said pharmaceutical composition may be used as a medicament, particularly in the treatment of stable angina pectoris and chronic heart failure.

### BRIEF DESCRIPTION OF THE DRAWINGS

FIG. 1 shows the full XRPD pattern of ivabradine hydrochloride polymorph IV. For measurement conditions see the Examples section.

### DETAILED DESCRIPTION OF THE PRESENT INVENTION

The present invention relates to a pharmaceutical composition comprising ivabradine hydrochloride polymorph IV, wherein the X-ray powder diffraction pattern of polymorph IV comprises characteristic peaks at the following 2 theta (±0.2) angles: 8.8°, 15.6°, 17.1°, 19.9°, 24.2° and 24.5°, measured using a Cu Kα radiation, characterized in that the composition is stabilized by a moisture barrier with a WVTR of less than 0.35 g/m²/day at 38° C./90% RH.

It was experienced in our laboratory that, especially within the pharmaceutical composition, polymorph IV, which is a hemihydrate, transforms over time into the more stable polymorph β, being a tetrahydrate. This transformation is even more pronounced in humid environments. Water vapor transmission rate (WVTR), also moisture vapor transmission rate (MVTR), is a measure of the passage of water vapor through a substance. Various techniques are available to measure WVTR, ranging from gravimetric techniques that measure the gain or loss of moisture by mass, to highly sophisticated instrumental techniques that in some designs can measure extremely low transmission rates. Commercial instruments are available to determine WVTRs using either a pressure-modulated infrared detector or a mechanically modulated infrared detector. Numerous standard methods are described in e.g. ISO, ASTM, BS and DIN, like ASTM F1249 and DIN53122. The conditions under which the measurement is made has a considerable influence on the result. Both the temperature and the humidity gradient across the sample need to be measured, controlled and recorded with the result. We have discovered that pharmaceutical compositions comprising ivabradine hydrochloride polymorph IV can be stabilized by a moisture barrier with a WVTR of less than 0.35 g/m²/day at 38° C./90% RH. Preferably, the WVTR of the moisture barrier is kept below 0.2 g/m²/day at 38° C./90% RH. More preferably, the WVTR is kept below 0.1 g/m²/day at 38° C./90% RH and most preferably, the WVTR is kept below 0.01 g/m²/day at 38° C./90% RH.

The pharmaceutical compositions of the present invention comprise ivabradine hydrochloride polymorph IV and one or more pharmaceutically acceptable excipients. The excipients to be used in accordance with the present invention are well-known and are those excipients which are conventionally used by the person skilled in the art. Depending on the dosage form chosen for the pharmaceutical composition, the person skilled in the art will be able to select suitable pharmaceutically acceptable excipients. Preferably, the dosage form is an immediate-release tablet and the pharmaceutically acceptable excipients are chosen from one or more binders, diluents, disintegrants, glidants, lubricants, stabilizers, surface active agents or pH-adjusting agents.

The present invention further comprises a process to prepare pharmaceutical compositions comprising ivabradine hydrochloride polymorph IV and one or more pharmaceutically acceptable excipients. The process comprises blending a composition of ivabradine hydrochloride polymorph IV with one or more pharmaceutically acceptable excipients, followed by compression of the blend into tablets, using equipment and methods well-known in the art.

The pharmaceutical compositions of the present invention display dissolution behavior typical for immediate-release formulations. During preparation and storage of the pharmaceutical compositions of the present invention, ivabradine hydrochloride remains in polymorphic form IV.

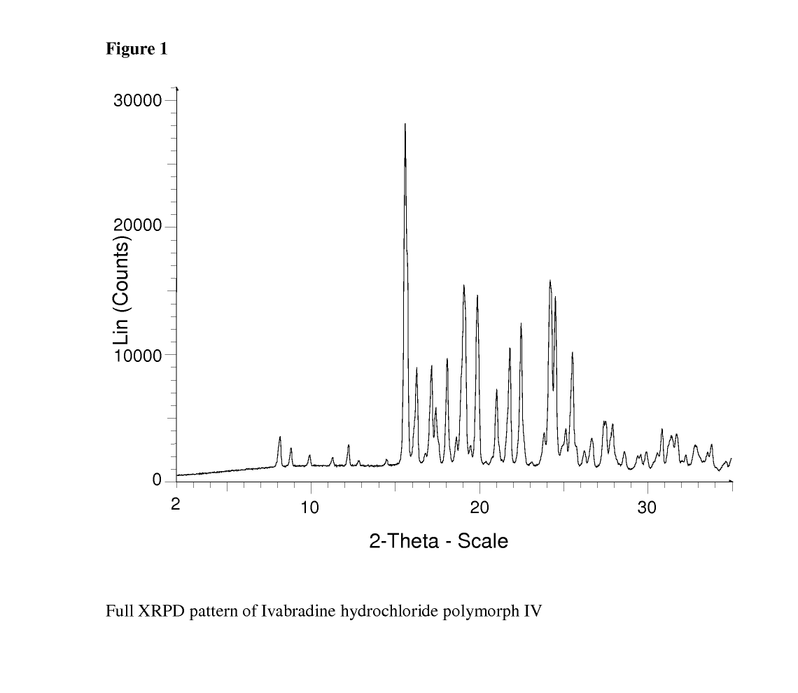

FIG. 1 shows the full X-ray powder diffraction pattern of ivabradine hydrochloride polymorph IV. Since the amount of ivabradine hydrochloride in the pharmaceutical composition is very small and since excipients are interfering in the analysis, X-ray powder diffraction appeared to be unsuitable as analytical technique to determine polymorphism in the pharmaceutical composition. Instead, Transmission Raman was used, which turned out to be a powerful technique.

In a preferred embodiment of the invention, pharmaceutical compositions comprising ivabradine hydrochloride polymorph IV are stabilized by a moisture barrier created by means of packaging the tablet in blister pack material. Blisters belong to the group of primary packaging material in pharmaceutical industry. The WVTR value is a known parameter to express the passage of water vapor through the packaging material in order to control the required quality and shelf life. The type of blister pack material used and its film thickness, determines the WVTR value. Typical WVTR values of a 250 μm PVC blister film and of a duplex 250 μm PVC/90 g/m² PVDC blister film are >3.0 g/m²/day and 0.35 g/m²/day at 38° C./90% RH respectively. These WVTR values are determined according ASTM F1249 (“Water Vapor Transmission Rate through plastic film and sheeting using a modulated infrared sensor”). Cold Form Foil (CFF), also known as Alu-Alu foil, has a WVTR value of 0.005 g/m²/day at 38° C./90% RH. In the present invention, pharmaceutical compositions comprising ivabradine hydrochloride polymorph IV are stabilized by a moisture barrier with a WVTR of less than 0.35 g/m²/day at 38° C./90% RH. Preferably, the blister pack material is Cold Form Foil (CFF).

In another embodiment of the invention, pharmaceutical compositions comprising ivabradine hydrochloride polymorph IV are stabilized by a moisture barrier created by means of coating the tablet with a moisture barrier coating creating a moisture barrier with a WVTR of less than 0.35 g/m²/day at 38° C./90% RH. Various commercial coating systems are available to protect moisture sensitive drugs to increase physical and chemical stability. Non aqueous polymeric coating systems deal with regulatory restrictions, cumbersome procedures, pollution, etc. Sugar coatings have the disadvantage of having long processing time and high water influx during the coating process. In the present invention, pharmaceutical compositions comprising ivabradine hydrochloride polymorph IV are stabilized by means of coating the tablet with an aqueous coating system. The WVTR of the moisture barrier created by the coating is influenced by the choice of the type of coating system, but also by the coating thickness applied. Coatings are applied by using methods well known by the person skilled in the art. The amount of coating applied will depend on several factors, including the substrate to be coated and the equipment used to apply the coating. By preparing a cast film with a specific thickness (e.g. 100 microns) and using for example a VTI WPA-100 apparatus, the WVTR value of a coating can be determined. Most preferably, the aqueous coating system is Opadry® 200 or Opadry® amb.

In another embodiment of the invention, pharmaceutical compositions comprising ivabradine hydrochloride polymorph IV are stabilized by a moisture barrier created by combining the aforementioned means. In other words, the pharmaceutical compositions comprising ivabradine hydrochloride polymorph IV are stabilized by a moisture barrier created by means of packaging the tablet in blister pack material in combination with coating the tablet with a moisture barrier coating creating a moisture barrier with a WVTR of less than 0.35 g/m²/day at 38° C./90% RH.

In yet another embodiment of the invention, the pharmaceutical compositions comprising ivabradine hydrochloride polymorph IV are stabilized by a moisture barrier by any of the aforementioned means, or a combination thereof, and wherein in addition one or more non-hygroscopic excipients and/or one or more moisture scavengers are used. A preferred example of a non-hygroscopic excipient is mannitol. Mannitol is widely used in pharmaceutical compositions. It is primarily used as diluent in tablet formulations, where it is of particular value since it is not hygroscopic and may thus be used with moisture-sensitive active pharmaceutical ingredients. A preferred example of an excipient that can act as moisture scavenger is Starch 1500®. Starch 1500® is partially pregelatinized maize starch and is a multifunctional excipient combining several properties: binder, disintegrant, filler and flow-aid while having lubricant properties. Starch 1500® is particularly effective as scavenger for the protection of moisture sensitive active pharmaceutical ingredients. Another preferred example of a moisture scavenger is silica, also known as silicon dioxide. Silica is porous and adsorbs moisture very well.

The pharmaceutical composition in accordance with the present invention may be used as a medicament. The pharmaceutical composition typically may be used in the treatment of stable angina pectoris and chronic heart failure.

The present invention is illustrated by the following Example.

### EXAMPLES

The full XRPD pattern of Ivabradine hydrochloride polymorph IV of FIG. 1 was obtained using a Bruker-AXS D8 Vario diffractometer with θ/2θ geometry (reflection mode), equipped with a Vantec PSD detector and applying the following measurement conditions:

Start angle (2θ): 2.0°
End angle (2θ): 35.0°
Scan step width: 0.02°
Scan step time: between 0.2-2.0 seconds
Radiation type: Cu
Radiation wavelengths: 1.5406 Å (Kα1), primary monochromator used
Exit slit: 6.0 mm
Focus slit: 0.2 mm
Divergence slit: Variable (V20)
Antiscatter slit: 11.8 mm
Receiving slit: 20.7 mm

### Example 1

### Preparation of 2.5 mg Ivabradine Hydrochloride Polymorph IV Tablets (Coated and Uncoated) and Subsequent Stability Study in Different Blister Pack Materials

Preparation of Ivabradine Hydrochloride Polymorph IV Tablets

51.64 g Ivabradine hydrochloride polymorph IV was mixed with 7.5 g Aerosil 200 VV and 200 g lactose for 10 minutes in a Turbula mixer at 22 rpm. The pre-mix was sieved over a 0.5 mm sieve. 933.33 g Lactose and 300 g maize starch were added and the resulting mixture was mixed for 15 minutes in a Bohle mixer at 25 rpm. The blend was sieved over a Comill 813 sieve and the obtained blend was mixed in a Bohle mixer for 20 minutes at 25 rpm. 7.5 g Magnesium stearate was sieved over a 0.6 mm sieve and was added to the blend. The blend was mixed in a Bohle mixer for 5 minutes at 25 rpm. Tablets were compressed using a Korsch ph 106 with forced feeder, mass 80 mg, 6 mm punch.

The tablets were analyzed by Transmission Raman (Laser: 830 nm; Power at sample: >600 mW; Fixed Grating: 730 g/mm). The spectra are in accordance with crystalline ivabradine hydrochloride polymorph IV.

The tablets showed a fast dissolution at pH 1.2.

Coating of Ivabradine Hydrochloride Polymorph IV Tablets

24 g Opadry II (not known as moisture protective coating) was dispersed in 136 g water. 800 g of 2.5 mg ivabradine hydrochloride polymorph IV tablets, obtained as described in example 1, were coated with the coating suspension in a Bohle drum coater using a divider disc.

The tablets were analyzed by Transmission Raman (Laser: 830 nm; Power at sample: >600 mW; Fixed Grating: 730 g/mm). The spectra are in accordance with crystalline ivabradine hydrochloride polymorph IV.

The tablets showed a fast dissolution at pH 1.2.

Stability Study of Ivabradine Hydrochloride Polymorph IV Tablets (Uncoated and Coated) in Different Blister Pack Materials

The uncoated tablets as obtained in example 1 and the coated tablets as obtained in example 2 were packed in three different blister pack materials: 250 μm PVC blister film, duplex 250 μm PVC/90 g/m² PVDC blister film and Cold Form Foil, also known as Alu-Alu foil.

The packed tablets were stored at 40° C./75% RH and the samples were analyzed by Transmission Raman (Laser: 830 nm; Power at sample: >600 mW; Fixed Grating: 730 g/mm).

Results Uncoated Tablets

| Blister pack | | |
|---|---|---|
| material | Time point | Conclusion |
| PVC | 2.5 months at 40° C./75% RH | Mainly ivabradine |
| | | hydrochloride |
| | | polymorph β |
| Duplex | 6 months at 40° C./75% RH | Ivabradine hydrochloride |
| | | polymorph β detected |
| CFF | 6 months at 40° C./75% RH | Ivabradine hydrochloride |
| | | polymorph IV; no |
| | | polymorph β detected |

Results Coated Tablets

| Blister pack | | |
|---|---|---|
| material | Time point | Conclusion |
| PVC | 2.5 months at 40° C./75% RH | Mainly ivabradine |
| | | hydrochloride polymorph β |
| Duplex | 6 months at 40° C./75% RH | Ivabradine hydrochloride |
| | | polymorph β detected |
| CFF | 6 months at 40° C./75% RH | Ivabradine hydrochloride |
| | | polymorph IV; |
| | | no polymorph |
| | | β detected |

1. A pharmaceutical composition comprising ivabradine hydrochloride polymorph IV, wherein the X-ray powder diffraction pattern of polymorph IV comprises characteristic peaks at the following 2 theta (±0.2) angles: 8.8°, 15.6°, 17.1°, 19.9°, 24.2°and 24.5°, measured using a Cu Kα radiation, characterized in that the composition is stabilized by a moisture barrier with a WVTR of less than 0.35 g/m²/day at 38° C./90% RH.

2. A composition according to claim 1, being an immediate-release tablet comprising ivabradine hydrochloride and pharmaceutically acceptable excipients, chosen from one or more binders, diluents, disintegrants, glidants, lubricants, stabilizers, surface active agents or pH-adjusting agents.

3. A composition according to claim 1, wherein the moisture barrier is created by means of packaging the tablet in blister pack material.

4. A composition according to claim 3, wherein the blister pack material is Cold Form Foil.

5. A composition according to claim 1, wherein the moisture barrier is created by means of coating the tablet with a moisture barrier coating.

6. A composition according to claim 5, wherein the moisture barrier coating is Opadry® 200 or Opadry® amb.

7. A composition according to claim 1, wherein the moisture barrier is created by combining the means according to anyone of claims 3 to 6.

8. A composition according to claim 1, wherein the moisture barrier is created by the means according to anyone of claims 3 to 6 or a combination thereof, and wherein in addition one or more non-hygroscopic excipients and/or one or more moisture scavengers are used.

9. A composition according to claim 8, wherein the non-hygroscopic excipient is mannitol.

10. A composition according to claim 8, wherein the moisture scavenger is Starch 1500®.

11. A composition according to claim 8, wherein the moisture scavenger is silica.

12. A process for preparing the pharmaceutical composition according to claim 1 comprising blending ivabradine hydrochloride polymorph IV with one or more pharmaceutically acceptable excipients, followed by compressing the blend into tablets.

13. The composition according to claim 1, for use as a medicament.

14. The composition according to claim 13 for use in the treatment of stable angina pectoris and chronic heart failure.
